(19) United States
(12) Patent Application Publication
(10) Pub. No.: US 2022/0304759 A1
(43) Pub. Date: Sep. 29, 2022

(54) MEDICAL CART FOR USE IN PATIENT CARE

(71) Applicant: Dominion Investments LLC, Salt Lake City, UT (US)

(72) Inventors: Steven Jay HANSEN, Round Rock, TX (US); Richard E. OLSEN, Salt Lake City, UT (US)

(73) Assignee: Dominion Investments LLC, Salt Lake City, UT (US)

(21) Appl. No.: 17/693,143

(22) Filed: Mar. 11, 2022

(30) Foreign Application Priority Data
Mar. 25, 2021 (CA) 3113262

(51) Int. Cl. A61B 50/13; A61G 12/00; A61M 5/14; B62B 3/02; B62B 5/00; B62B 3/00; B60B 33/04

(52) U.S. Cl. CPC A61B 50/13; A61G 12/001; A61M 5/1415; B62B 3/02; B62B 5/00; B62B 3/005; B60B 33/045; B62B 3/104

(57) ABSTRACT

A medical cart for use in patient care is provided, the medical cart comprising: a front; a back; sides; four legs which have a distal end, a proximal end and a bore at least partially therebetween terminating in an aperture at the proximal end; a spring plunger mounted on each leg for releasably retaining a pole in the bore; a bracket attached to each leg proximate to the distal end; a caster rotatably attached to each bracket; a lower shelf attached to the legs and extending between the legs; a ballast sleeve below the lower shelf for releasably retaining weight plates; and an upper shelf attached to the legs and extending between the legs, the upper shelf including a recess at the front extending towards the back.

### CROSS REFERENCE TO RELATED APPLICATIONS

The present invention is related to and claims the benefit of Canadian Application Serial No. 3,113,262, filed on Mar. 25, 2021, and entitled MEDICAL CART FOR USE IN PATIENT CARE and which is hereby incorporated in its entirety including all tables, figures, and claims.

### FIELD

The present technology is a highly stable and mobile medical cart which is configurable for a range of capabilities. More specifically it is a medical cart that can carry multiple pole attachments including intravenous (IV poles), multiple e-size cylinders, for example oxygen tanks and other patient specific equipment and is designed to stay with a patient during surgery and follow them throughout their stay in the hospital.

### BACKGROUND

There are many designs of medical carts, many of which are purpose built and offer little flexibility in what they hold or carry. For example, United States Patent Application Publication No. 20200222261 discloses a medical cart that allows confirmation that individual medicines have been managed at appropriate temperatures. The medical cart includes: shelves on which medicines can be placed; and a control part which communicates with an RF tag provided for each of the medicines and equipped with a temperature sensor, at appropriate time intervals via an antenna part provided in the vicinity of each of the shelves, and the control part has a memory part which stores ID information of the RF tag and temperature information sensed by a temperature sensor of the RF tag. The medical cart is not configurable for different tasks. Further, the cart is not self-leveling. Still further, the casters are disposed underneath the rest of the cart, thus not offering stability to the cart.

United States Patent Application Publication No. 20190105119 discloses a medical cart having a chassis with a plurality of wheels attached thereto; a plurality of drawers supported on the chassis; a temperature-regulated compartment in the chassis; a locking bar pivotally attached to the chassis; a headwall mount attached to the chassis; a display mounted on the chassis; and a positionable task light attached to the chassis. Each drawer includes a drawer lock shiftable between a locked position and an unlocked position. The locking bar is shiftable between a locked position and an unlocked position so that the locking bar serves as a secondary safety lock for all the drawers and the door of the temperature-regulated compartment. The medical cart is not configurable for different tasks. Further, the cart is not self-leveling.

United States Patent Application Publication No. 20180319214 discloses a swiveling caster wheel system for use on rolling devices such as children's accessories, furniture or equipment, and including a locking system having a locking actuator that is fixed in position regardless of the swivel position of the caster wheel. The system allows a user to access and actuate the caster locking system from a consistent position of the rolling device regardless of the swivel position of the caster wheel and allows the caster wheel to be covered without blocking access to the locking actuator. The wheels do not permit the cart to self-level. It is not designed for multiple caster and suspension options.

United States Patent Application Publication No. 20160022526 discloses a mobile cart that includes a cabinet, wheels, drawers and an illumining unit. The wheels are disposed on a bottom of the cabinet. The drawers are disposed in the cabinet and configured to be pulled out of the cabinet. The drawers include compartment drawers. The illumining unit is disposed on a top of the cabinet and includes a compartment drawer illumining module, wherein the compartment drawer illumining module is configured to illumine any one of the compartment drawers being pulled out of the cabinet. The medical cart is not configurable for different tasks. Further, the cart is not self-leveling. It is not designed for multiple caster and suspension options. Still further, the wheels are disposed underneath the rest of the cart, thus not offering stability to the cart.

United States Patent Application Publication No. 20150305086 discloses a medical cart that includes an interface assembly including a display, a speaker, a camera and a microphone. The interface assembly is operatively connected to an enclosure at a connecting portion that is operable to reposition the interface assembly from a transporting configuration in which the interface assembly is disposed inside the enclosure to a deployed configuration in which the interface assembly is disposed outside of the enclosure. The medical cart also includes a set of wheels operably connected to at least one of the enclosure or a pivoting assembly operable to pivot from the transporting configuration in which only the set of wheels makes contact with a transporting surface to the deployed configuration in which a portion of the cart in addition to the set of wheels makes contact with the transporting surface for stabilizing the cart in the deployed configuration. The medical cart is not configurable for different tasks. Further, the cart is not self-leveling although the cart can be raised and lowered by urging the wheels closer to the cart or farther away from the cart respectively. It is not designed for multiple caster and suspension options.

United States Patent Application Publication No. 20150166090 discloses medical carts and associated apparatuses and methods. A medical cart configured in accordance with one embodiment includes a cart base assembly, a work platform, and a height adjustment assembly carrying the work platform above the cart base assembly. The cart base assembly includes an upper portion and a touch-sensitive foot panel generally below the upper portion. The touch-sensitive foot panel includes a resilient cover having a contact surface and a contact switch underlying the resilient cover. The contact switch is operably coupled to the height adjustment assembly, and the contact switch is configured to detect for operator foot contact at the contact surface and to raise or lower the work platform via the height adjustment assembly when the operator foot contact is detected. The medical cart is not configurable for different tasks. Further, the cart is not self-leveling. It is not designed for multiple caster and suspension options.

United States Patent Application Publication No. 20140218282 discloses a medical cart includes a cart body, a top platform, and an elevating device. The elevating device is mounted on the cart body for supporting the top platform above the cart body and is for moving the top platform upwardly and downwardly. The elevating device includes a sleeve, a screw rod, a motor and a motor driving module. The screw rod extends through the sleeve and engages threadedly a threaded inner surface of the sleeve. The motor is connected to the screw rod and is for driving the screw rod to rotate and move with respect to the sleeve, such that the top platform is driven by the screw rod to move upwardly and downwardly with respect to the cart body. The medical cart is not configurable for different tasks. Further, the cart is not self-leveling. It is not designed for multiple caster and suspension options. Still further, the wheels are disposed underneath the rest of the cart, thus not offering stability to the cart.

United States Patent Application Publication No. 20130307237 discloses a medical cart system, which includes a base, a tower, a work station, a quick-release unit, and a drawer assembly. The base is supported by wheels, and the tower is disposed on the top surface of the base. The work station is movably disposed on the tower. The work station includes at least one work platform disposed at the front of the tower and at least one display arranged above the work platform. The quick-release unit is removably disposed on the tower. The drawer assembly includes one cabinet removably disposed on the tower. The cart is not self-leveling. It is not designed for multiple caster and suspension options. Further, the wheels are disposed underneath the rest of the cart, thus not offering stability to the cart.

United States Patent Application Publication No. 20050159784 discloses a medical cart, which includes respiration and cardiac functionality, that can readily be transported to a patient. The cart may contain separable respiratory and cardiac units in which the cardiac unit contains one or more items useful in treating cardiac emergencies such as cardiac arrest and the respiratory unit contains one or more items useful in treating respiratory emergencies such as a stoppage of breathing. To increase access to the items that the cart carries, parts of the cart, e.g., drawers, which contain items associated with particular medical conditions, may be isolated. As a result, multiple users, who are treating different medical conditions, can access the cart at any given time. A hood may be provided to enclose items, which were placed on top of the cart, while the cart is in transit. The medical cart is not configurable for different tasks. Further, the cart is not self-leveling. It is not designed for multiple caster and suspension options. Still further, the wheels are disposed underneath the rest of the cart, thus not offering stability to the cart.

What is needed is a highly stable and mobile medical cart that can accept a range of accessories including poles with IV hooks, gas line hooks, and hose hangers, infusion poles, leg extensions for a range of miscellaneous items, multiple e-size cylinders (e.g. oxygen tank), and a heater-cooler unit strap. It would preferably include self-adjusting or adjustable casters for leveling the cart. It would preferably include a recess region on the top for equipment to sit in or sit over.

### SUMMARY

The present technology is a stainless steel medical cart that is versatile, stable and highly mobile so it can move with a patient even if running down a hallway. The medical cart can accept a range of accessories including poles with IV hooks, gas line hooks, and hose hangers, infusion poles, leg extensions for a range of miscellaneous items, e-size cylinders (e.g. oxygen tanks), and a heater-cooler unit strap. It includes a tray for accepting ballast and has brackets on which the casters are mounted that extend out beyond the body of the medical cart, thus providing superior stability. The cart can either have no suspension, independent suspension or be self-leveling or leveled with drives that raise and lower the casters independently. The brackets that retain the suspension and the casters can be easily removed and exchanged. The medical cart has a recess on the top for equipment to sit in or sit over.

In one embodiment, a medical cart is provided for use in patient care, the medical cart comprising: a front; a back; sides; four legs which have a distal end, a proximal end and a bore at least partially therebetween terminating in an aperture at the proximal end; a spring plunger mounted on each leg for releasably retaining an accessory pole in the bore; a bracket attached to each leg proximate to the distal end; a caster rotatably attached to each bracket; a lower shelf in slidable engagement with the sides; a ballast tray below the lower shelf for releasably retaining weight plates; and an upper shelf which is attached to the side, the upper shelf including a recess at the front extending towards the back.

The medical cart may further comprise a releasable lock on the front for the lower shelf.

The medical cart may further comprise a lip around the upper shelf around the back and the sides.

In the medical cart, the recess and the lip of the upper shelf define a ledge along the sides.

The medical cart may further comprise a drawer slidably mounted below the upper shelf and opening to the back.

The medical cart may further comprise a thumb screw located below the spring plunger on each leg.

The medical cart may further comprise the at least one accessory pole, the accessory pole including a pole which includes apertures which releasably retain the spring plunger, releasably retaining the pole in the bore, the pole extending upward therefrom and terminating in a hanger block, which includes a plurality of hook slots and a threaded cap which threadedly engages the hanger block.

The medical cart may further comprise a plurality of hooks which are releasably retained in the plurality of hook slots.

The medical cart may further comprise a least one cylinder holder, the cylinder holder releasably mounted on at least one side.

In the medical cart, the brackets may be bolted onto the legs.

In the medical cart, the brackets may extend outward from the legs at an angle of about 45 degrees relative to the lower shelf.

The medical cart may further comprise suspension housed in each bracket.

In the medical cart, the suspension may be adjustable.

In the medical cart, the suspension may be a pneumatic shock, a hydraulic shock or be spring loaded.

In the medical cart the suspension may include a motor drive.

In the medical cart, the casters may be self-adjusting and the medical cart may further comprise at least one digital spirit level, a printed circuit board in electronic communication with the digital spirit level and at least one servo motor under control of the printed circuit board.

The medical cart may further comprise a handle, the handle attached to the sides and extending outward from the back.

In the medical cart, the handle may be foldable.

In another embodiment, an accessory pole is provided for use with a medical cart, the accessory pole comprising: a pole which includes a distal end, a proximal end and a length therebetween, the length including a plurality of apertures along a portion of the length; a hanger block connected to the proximal end, the hanger block including a top, a bottom and sides, a plurality of slots extending from the top along the sides and a threaded aperture in the top; and a threaded knob, the threaded knob releasably retained in the threaded aperture and covering the slots in the top.

In the accessory pole, the pole may include a terminal plate at a proximal end, the terminal plate releasably retaining the hanger block.

The accessory pole may further comprise a plurality of hooks or hangers, each hook or hanger releasably retained in a slot of the plurality of slots.

In the accessory pole each slot of the plurality of slots may be T-shaped.

### FIGURES

FIG. 1A is a perspective view of the cart of the present technology; and

FIG. 1B is taken along line B-B.

FIG. 2 is a partial side view of the cart of FIG. 1.

FIG. 3 is a side view of the spring plunger.

FIG. 4 is a side view of a caster with suspension.

FIG. 5 is a side sectional view of self-leveling caster.

FIG. 6 is a side sectional view of an alternative embodiment caster.

FIG. 7A is a side view of an accessory pole; FIG. 7B is a top perspective view of the hanger block of the accessory pole and FIG. 7C is a side view of a hanger or hook.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

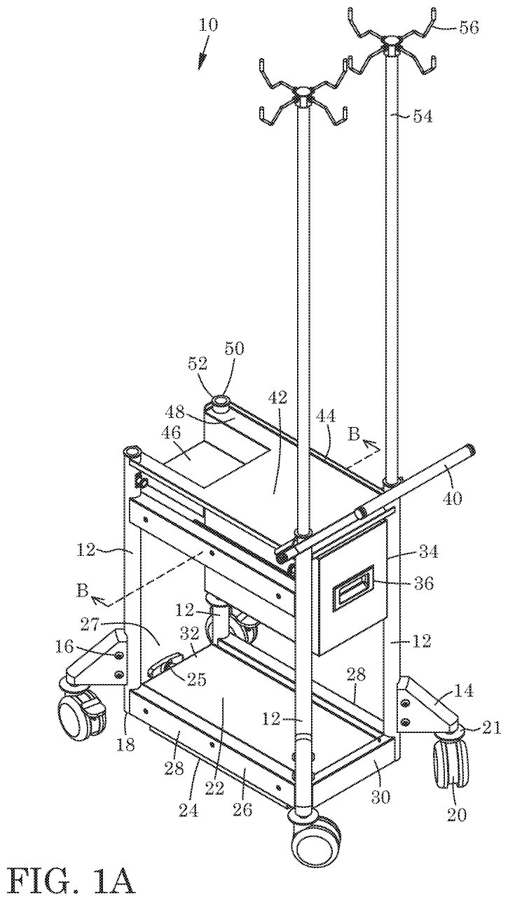

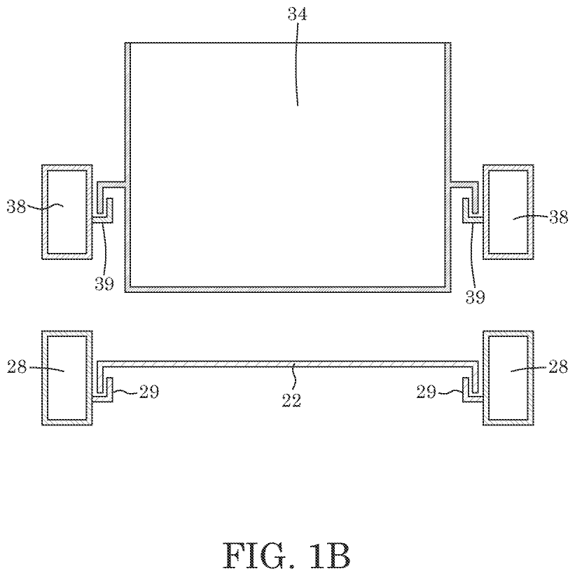

A medical cart, generally referred to as 10 is shown in FIG. 1A. It is made of stainless steel. The cart 10 has four legs 12 each with a bracket 14 mounted with bolts 16 to them. The back legs are taller than the front legs. Each bracket 14 is mounted proximate to the distal end 18 of the leg 12 and extends outward at about a 45 degree angle relative to the cart 10 to provide stability. A caster 20 is pivotally mounted to the bracket 14 with a caster adapter plate 21. The midline of the caster 20 is approximately level with the distal end 18 of the leg 12 thus providing a low profile. A lower shelf 22 is proximate to the distal end 18 of the legs 12 and is slidably attached to lower side braces 28 by drawer slides 29 (see FIG. 1B) which allow for the lower shelf 22 to be extended and retracted. A cam latch 25 holds the lower shelf 22 in place in the retracted position. The side braces 28 extend above the level of the lower shelf 22 as a lip 26 around the sides 28 and back 30 with an open front 32. A ballast tray 24 is located below the lower shelf 22. It is designed to accept up to three weight plates to provide further stability to the cart 10. The weight plates are bolted to the ballast tray 24. A drawer 34 with a pull 36 is slidably mounted on drawer slides 39 (see FIG. 1B) which are attached to upper side braces 38. The upper side braces 38 and the lower side braces 28 form the sides. In one embodiment, the upper side braces 38 and the lower side braces 28 are replaced with sides. The drawer 34 opens from the back 30 of the cart 10. An adjustable and foldable handle 40 extends out from the back 30. An upper shelf 42 is fixed to the legs 12. It has a lip 44 around the upper side and back 30 with an open front 32. A recess 46 extends from the front 32 towards the back 30. There is a ledge 48 between the lip 44 on the upper side and the recess 46. The ledge 48 and recess 46 are designed for equipment with knobs and controls located near the bottom of the equipment. The equipment may rest on the ledges 48 and on the upper shelf 42 and span the recess 46 making access much easier for a user. The legs 12 have a bore that terminates in an aperture 50 at the proximal end 52 of the legs 12. The bore extends at least a portion of the length of the leg 12. The aperture 50 and bore accept an accessory pole 54 or other accessories. As shown in FIG. 1 the accessory pole 54 is an IV pole and IV hooks 56 are releasably connected to the accessory pole 54. Other accessories and poles include gas line hooks, hose hangers, infusion poles, leg extensions for a range of miscellaneous items and the like. All four legs 12 can retain an accessory pole 54 or other accessory, for example: (1) the IV Poles; (2) the Long Leg Extensions, about 12″ long that medical personnel can clip miscellaneous things to; and (3) Short Leg Extensions, about 2″ long with hooks on them that can be used with or without straps to secure equipment to the cart. The standard configuration may include two IV Poles and two Short Leg Extensions with hooks.

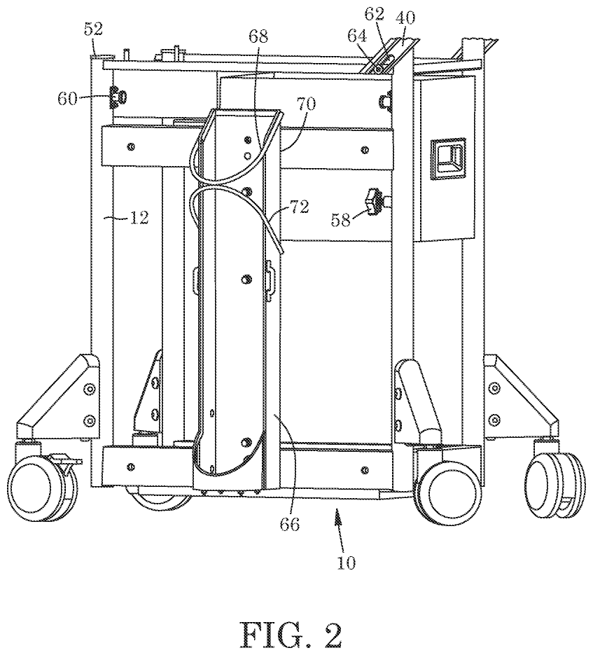

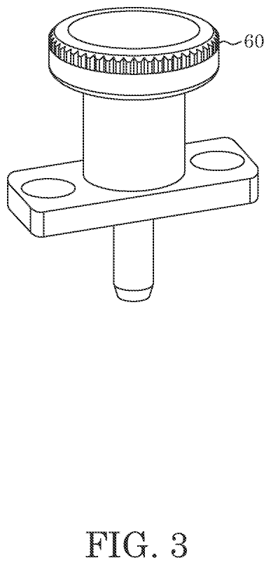

As shown in FIG. 2, a spring plunger 60 is located proximate to the proximal end 52 of the leg 12. It extends through an aperture into the bore to releasably retain the accessory pole 54. The details of the spring plunger 60 are shown in FIG. 3. Returning to FIG. 2, a thumb screw 58 is located below the spring plunger 60 and extends through an aperture into the bore to add further stability to the accessory pole 54. The handle 40 has a slot 62 that slidably accepts a bolt 64 to allow for adjustment of the handle 40. The handle is foldable. An e-size cylinder holder 66 is bolted to braces 38 and 28 (see FIG. 1) on either or both sides of the cart 10. An upper hoop 68 is attached to the side 70 of the e-size cylinder holder 66 and a lower hoop 72 is pivotally attached to the side 70 of the e-size cylinder holder 66. The e-size cylinder holder 66 typically retains an oxygen cylinder.

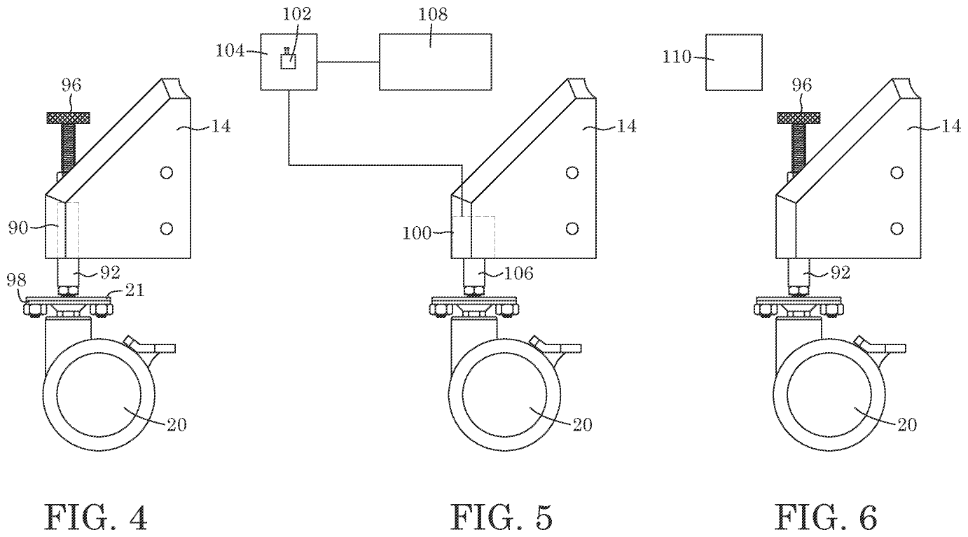

In one alternative embodiment shown in FIG. 4, each caster 20 has suspension to move up and down over uneven surfaces while keeping the body of the cart 10 stable and level. A spring 90 is mounted inside the bracket 14 and is attached to a shaft 92 that extends from the bracket 14 to a caster adapter plate 21. A thumb screw 96 extends from the shaft 92 through the bracket 14 and into the ambient environment for manually adjusting the recoil on a given caster to accommodate a heavier load on that section of the cart 10. The caster adapter plate 21 is attached to a caster mounting plate 98. In an alternative embodiment the spring 90 and shaft 92 are replaced with a hydraulic or pneumatic ram.

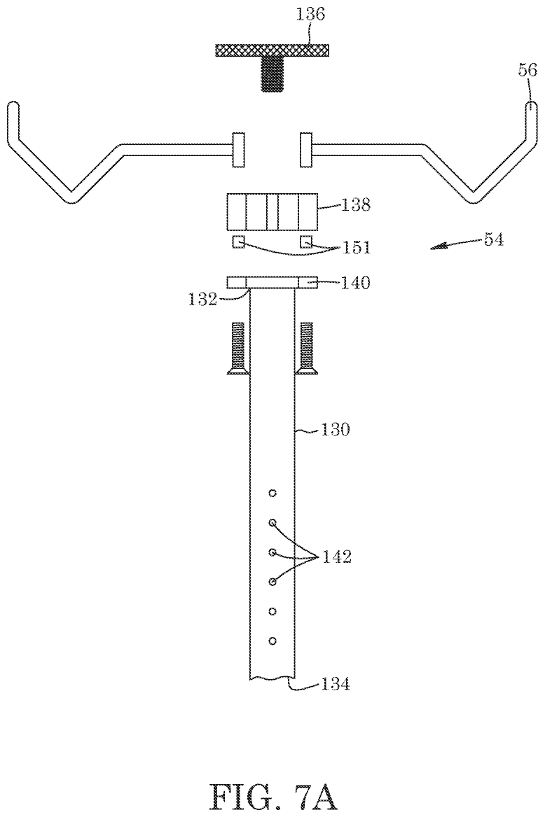

In another alternative embodiment shown in FIG. 5, the casters 20 are self-leveling. The casters 20 are automatically adjusted when the cart 10 is stationary. Each caster 20 has a servo motor 100 associated with it and a digital spirit level 102 integrated with a printed circuit board (PCB) 104 on the cart 10 that signals the servo motors 100 to bring the cart 10 to level once it is in position. The servo motor 100 is housed in the bracket 14 and the integrated digital spirit level 102 and PCB 104 is housed on the cart, for example under the center of the upper shelf 42. The servo motor 100 is mechanically linked to the shaft 106 and in electronic communication with the printed circuit board 104. The shaft 106 extends from the bracket 14 and is attached to the caster adapter plate 21. The caster adapter plate 21 is attached to a caster mounting plate 98. A battery 108 powers the system.

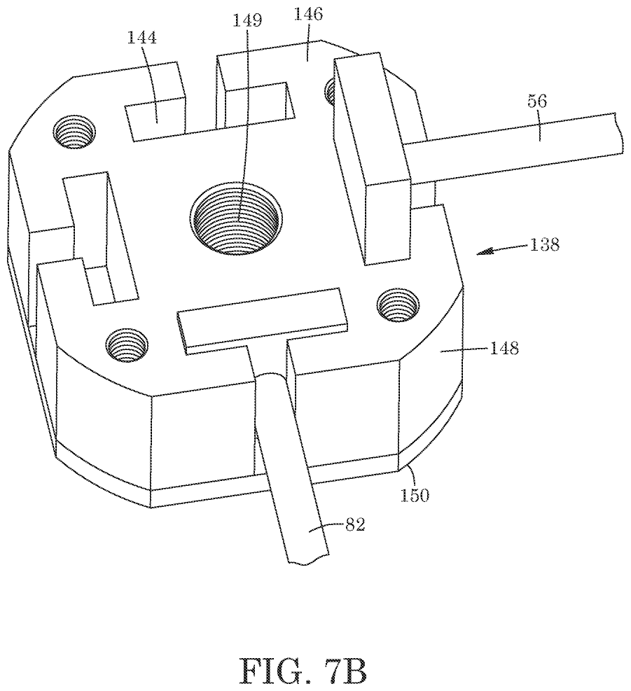

In another embodiment, shown in FIG. 6, each caster 20 is manually adjustable and there are at least two visual spirit levels 110, located, for example on top of the upper side brace 38 for easy reference by the user. A thumb screw 96 extends from the shaft 92 through the bracket 14 and into the ambient environment for manually adjusting the casters 20. The various caster designs can be installed simply by unbolting the brackets 14 that hold the casters 20 in place.

A bracket may be attached to the medical cart 10 to allow the cart to be attached to a medical bed, thus moving the bed and cart can be done by one person.

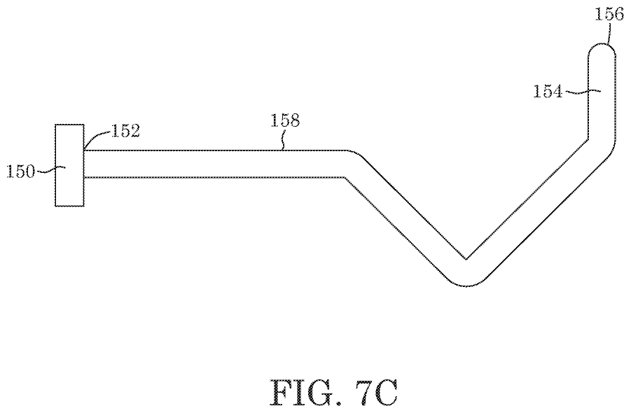

As shown in FIG. 7A, the hook or hanger 56 and the accessory pole 54 together provide an accessory assembly, generally referred to as 128. The poles 130 of the accessory poles 54 have a distal end 132 and a proximal end 134. The accessory poles 54 terminate in a threaded cap 136 which has a knurled side. Below the threaded cap 136 is a hanger block 138 for accepting a hook or hanger 56. The hanger block 138 is bolted onto a terminal plate 140 which is welded onto each accessory pole 54 at the distal end 132 of the pole 130. The pole 130 has three or more apertures 142 that engage the spring plunger 60, releasably retaining the accessory poles 54 in place.

As shown in FIG. 7B, the hooks 56 slide into T-shaped hook slots 144 which are in the top 146 and extend a distance down the sides 148 towards the bottom 150 in the hanger block 138. The hooks 56 are releasably retained with the threaded cap 136, which is releasably retained in the threaded aperture 149. They remain in place when a user removes the cap 136 and can be easily removed by sliding them upward. Up to four hooks or hangers 56 can be retained on each of the four accessory poles 54. Returning to FIG. 7A, cord stock 151 is disposed in the T-shaped slots 144 and is compressed against the terminal plate 140 to provide a compressible surface that promotes retention of each hook 56 by the threaded cap 136.

As shown in FIG. 7C, the hanger 56 includes a block 150 at a terminal end 152, a hook 154 at the other end 156 and shaft 158 therebetween. The block 150 and the terminal end 152 of the shaft mate with the T-shaped slot 144. The threaded cap 136 covers at least the block 150 and preferably the top 146 of the hanger block 138 when in place. The hook or hanger 56 and the accessory pole 54 together provide an accessory assembly.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A medical cart for use in patient care, the medical cart comprising: a front; a back; sides; four legs which have a distal end, a proximal end and a bore at least partially therebetween terminating in an aperture at the proximal end; a spring plunger mounted on each leg for releasably retaining an accessory pole in the bore; a bracket attached to each leg proximate to the distal end; a caster rotatably attached to each bracket; a lower shelf in slidable engagement with the sides; a ballast tray below the lower shelf for releasably retaining weight plates; and an upper shelf retained by the sides.

2. The medical cart of claim 1, further comprising a releasable lock on the front for the lower shelf.

3. The medical cart of claim 2, further comprising a lip around the upper shelf around the back and the sides.

4. The medical cart of claim 3, further comprising a drawer slidably mounted below the upper shelf and opening to the back.

5. The medical cart of claim 4, further comprising a thumb screw located below the spring plunger on each leg.

6. The medical cart of claim 5 further comprising the at least one accessory pole, the accessory pole including a pole which includes apertures which releasably retain the spring plunger, releasably retaining the pole in the bore, the pole extending upward therefrom and terminating in a hanger block, which includes a plurality of hook slots and a threaded cap which threadedly engages the hanger block.

7. The medical cart of claim 6 further comprising a plurality of hooks which are releasably retained in the plurality of hook slots.

8. The medical cart of claim 7, further comprising at least one e-size cylinder holder, the e-size cylinder holder releasably mounted on either or both sides.

9. The medical cart of claim 8, wherein the brackets are bolted onto the legs.

10. The medical cart of claim 9, wherein the brackets extend outward from the legs at an angle of about 45 degrees relative to the lower shelf.

11. The medical cart of claim 10, further comprising suspension between each caster and each bracket.

12. The medical cart of claim 11, wherein the suspension is adjustable.

13. The medical cart of claim 12, wherein the suspension is a pneumatic or hydraulic ram or is spring loaded.

14. The medical cart of claim 13, where the upper shelf includes a recess at the front extending towards the back and the recess and the lip of the upper shelf define a ledge along the sides.

15. The medical cart of claim 14, wherein the casters are self-adjusting and the medical cart further comprises at least one digital spirit level, a printed circuit board in electronic communication with the digital spirit level and at least one servo motor under control of the printed circuit board.

16. The medical cart of claim 15, further comprising a handle, the handle attached to the sides and extending outward from the back.

17. The medical cart of claim 16, wherein the handle is foldable.

18. An accessory pole for use with a medical cart, the accessory pole comprising: a pole which includes a distal end, a proximal end and a length therebetween, the length including a plurality of apertures along a portion of the length; a hanger block connected to the proximal end, the hanger block including a top, a bottom and sides, a plurality of slots extending from the top along the sides and a threaded aperture in the top; and a threaded knob, the threaded knob releasably retained in the threaded aperture and covering the slots in the top.

19. The accessory pole of claim 18, wherein the pole includes a terminal plate at a proximal end, the terminal plate releasably retaining the hanger block.

20. The accessory pole of claim 19, further comprising a plurality of hooks or hangers, each hook or hanger releasably retained in a slot of the plurality of slots.

21. The accessory pole of claim 20 wherein each slot of the plurality of slots is T-shaped.
